(19) United States
(12) Patent Application Publication
(10) Pub. No.: US 2026/0241142 A1
(43) Pub. Date: Aug. 20, 2026

(54) DEFLECTABLE MEDICAL DEVICES

(71) Applicant: Boston Scientific Scimed, Inc., Maple Grove, MN (US)

(72) Inventors: Andrea M. Goering, Blaine, MN (US); Nicholas Lee Tassoni, Andover, MN (US); Sara Reda Eskander, Maple Grove, MN (US); Cooper Whalen Johnson, Champlin, MN (US); Carly Joan Rogers, Eden Prairie, MN (US)

(73) Assignee: Boston Scientific Scimed, Inc., Maple Grove, MN (US)

(21) Appl. No.: 19/541,861

(22) Filed: Feb. 17, 2026

Related U.S. Application Data
(60) Provisional application No. 63/759,754, filed on Feb. 18, 2025.

(51) Int. Cl. A61M 25/01; A61M 25/00

(52) U.S. Cl. CPC A61M 25/0141; A61M 25/0012; A61M 25/0045; A61M 25/0052

(57) ABSTRACT

Deflectable medical devices as well as methods for manufacturing deflectable medical devices are disclosed. An example deflectable medical device may include an elongate shaft having a distal end region. The distal end region may include a first polymer region defined along a first side. The first polymer region may include a first polymer. The distal end region may include a second polymer region defined along a second side, and the second polymer region may include a second polymer. The first polymer may have a different stiffness than the second polymer. The distal end region may be configured to have a first radius of curvature along the first polymer region when bending in a first direction and a second radius of curvature along the second polymer region when bending in a second direction.

### CROSS REFERENCE TO RELATED APPLICATIONS

This application claims the benefit of U.S. Provisional Patent Application Serial No. 63/759,754 filed on February 18, 2025, the disclosure of which is incorporated herein by reference.

### TECHNICAL FIELD

The present disclosure pertains to medical devices, and methods for manufacturing medical devices. More particularly, the present disclosure pertains to deflectable medical devices

### BACKGROUND

A wide variety of medical devices have been developed for medical use, and more specifically for intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A deflectable medical device is disclosed. The deflectable medical device comprises: an elongate shaft having a distal end region; wherein the distal end region has a first polymer region defined along a first side, the first polymer region including a first polymer; wherein the distal end region has a second polymer region defined along a second side, the second polymer region including a second polymer; wherein the first polymer has a different stiffness than the second polymer; and wherein the distal end region is configured to have a first radius of curvature along the first polymer region when bending in a first direction and a second radius of curvature along the second polymer region when bending in a second direction.

Alternatively or additionally to any of the embodiments above, the first direction and the second direction lie within a first plane.

Alternatively or additionally to any of the embodiments above, the first direction lies within a first plane and the second direction lies within a second plane different from the first plane.

Alternatively or additionally to any of the embodiments above, the elongate shaft includes an inner layer, a reinforcing layer, and an outer layer.

Alternatively or additionally to any of the embodiments above, the first polymer region is defined by replacing a first discrete section of the outer layer with the first polymer.

Alternatively or additionally to any of the embodiments above, the second polymer region is defined by replacing a second discrete section of the outer layer with the second polymer.

Alternatively or additionally to any of the embodiments above, further comprising a stiffening member disposed along the first polymer region.

Alternatively or additionally to any of the embodiments above, the stiffening member engages the reinforcing layer.

Alternatively or additionally to any of the embodiments above, the first polymer region includes a plurality of polymers.

Alternatively or additionally to any of the embodiments above, the first polymer region is formed by laser ablating an ablated region of the elongate shaft and disposing the first polymer along the ablated region.

A deflectable medical device is disclosed. The deflectable medical device comprises: an elongate shaft having a distal end region; wherein the elongate shaft includes an inner layer, a reinforcing layer, and an outer layer; wherein a first discrete section of the outer layer along the distal end region is replaced by a first polymer to define a first polymer region; wherein a second discrete section of the outer layer along the distal end region is replaced by a second polymer to define a second polymer region; wherein the first polymer has a different stiffness than the second polymer; and wherein the distal end region is configured to have a first radius of curvature along the first polymer region when bending in a first direction and a second radius of curvature along the second polymer region when bending in a second direction.

Alternatively or additionally to any of the embodiments above, the first direction and the second direction lie within a first plane.

Alternatively or additionally to any of the embodiments above, the first direction lies within a first plane and the second direction lies within a second plane different from the first plane.

Alternatively or additionally to any of the embodiments above, further comprising a stiffening member disposed along the first polymer region.

Alternatively or additionally to any of the embodiments above, the stiffening member is disposed along the first polymer region.

Alternatively or additionally to any of the embodiments above, the stiffening member engages the reinforcing layer.

Alternatively or additionally to any of the embodiments above, the first polymer region includes a plurality of polymers.

Alternatively or additionally to any of the embodiments above, the first discrete section of the outer layer is laser ablated from the outer layer.

A method for forming a deflectable medical device is disclosed. The method comprises: replacing a first discrete section of an outer layer of an elongate shaft with a first polymer to define a first polymer region; wherein the elongate shaft includes a distal end region, an inner layer, a reinforcing layer, and the outer layer; wherein the first discrete section of the outer layer is disposed along the distal end region of the elongate shaft; replacing a second discrete section of the outer layer along the distal end region with a second polymer to define a second polymer region; wherein the first polymer has a different stiffness than the second polymer; and wherein the distal end region is configured to have a first radius of curvature along the first polymer region when bending in a first direction and a second radius of curvature along the second polymer region when bending in a second direction.

Alternatively or additionally to any of the embodiments above, further comprising disposing a stiffening member adjacent to the first polymer region.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view schematically depicting an example deflectable medical device.

FIG. 2 is a cross-sectional view taken through line 2—2 in FIG. 1.

FIG. 3 is a side view of an example deflectable medical device.

FIG. 4 is a side view of an example deflectable medical device.

FIG. 5 is a cross-sectional view of an example deflectable medical device.

FIG. 6 is a cross-sectional view of an example deflectable medical device.

FIG. 7 is a cross-sectional view of an example deflectable medical device.

FIG. 8 is a cross-sectional view of an example deflectable medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

A number of medical interventions may require a clinician to navigate a medical device into the heart. For example, treatment of the left atrial appendage by implanting an occlusive device (e.g., WATCHMAN FLX™ or WATCHMAN FLX™ Pro from Boston Scientific of Marlborough, MA. USA) may require that the device has sufficient reach and/or the ability to navigate with tight deflection/maneuverability. If the intervention results in the clinician performing a pulmonary vein isolation (PVI) or PVI+ (PVI plus adjuvant atrial ablations) maneuver, which may include septal line, mitral isthmus ablation, and/or the like, even further reach and/or tighter deflections may be desired. In some instances, a clinician may perform a primary intervention (e.g., left atrial appendage occlusion) using a first sheath. In order to perform a secondary intervention (e.g., PVI and/or a PVI+ maneuver), a clinician may choose to remove the first sheath and replace with a second sheath with different properties such as different reach and/or different deflection properties. Disclosed herein are sheaths, for example steerable sheath, that have desirable levels of reach as well as the ability to navigate with tight deflection/maneuverability. Such sheaths may allow a clinician to perform a number of different medical interventions without needing to replace the sheath.

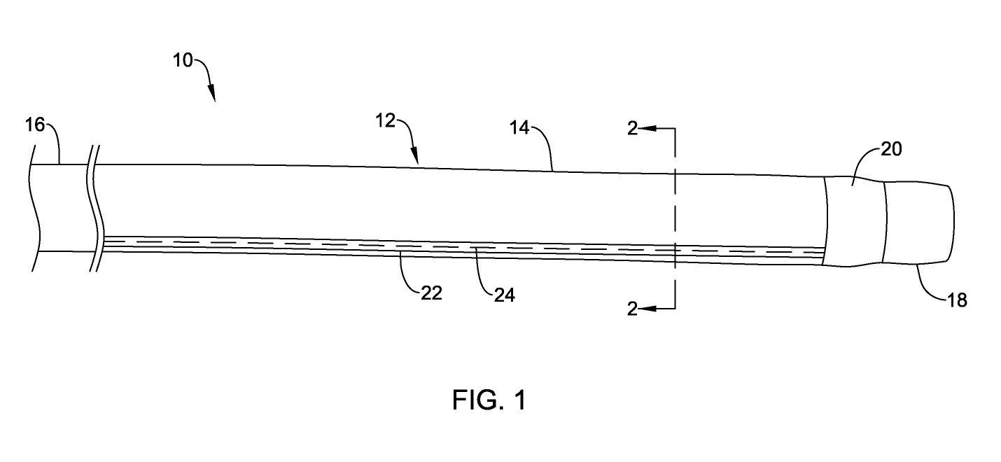

FIG. 1 schematically illustrates an example deflectable medical device 10. The deflectable medical device 10 may include an elongate shaft 12 having a distal end region 14 and a proximal end region 16 disposed proximal of the distal end region 14. The elongate shaft 12 may include a distal tip 18. The elongate shaft 12 may be steerable. As such, the elongate shaft 12 may include a steering member 20. A steering wire 24 may be coupled to the steering member 20. Actuating the steering wire 24 (e.g., pulling the steering wire 24) may cause the distal end region 14 to curve or bend. In FIG. 1, a singular steering wire 24 is depicted enabling unidirectional bending. Alternative elongate shafts are contemplated that include two or more steering wires 24, enabling bidirectional or multidirectional steering (including steering in multiple planes).

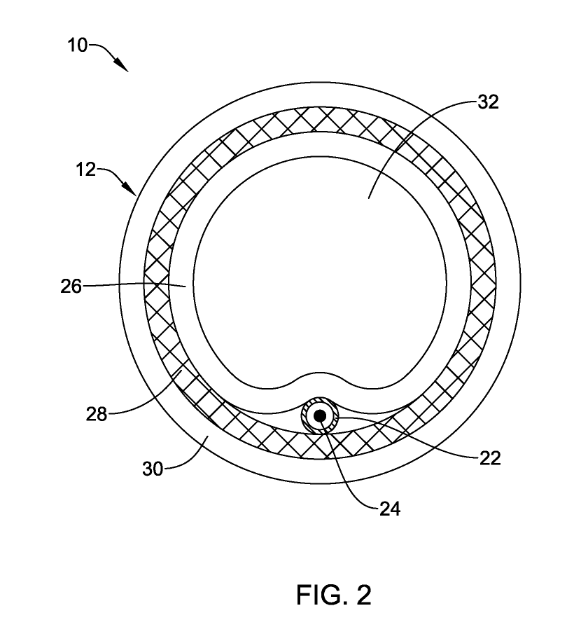

FIG. 2 is a cross-sectional view of the elongate shaft 12. Here the form of the elongate shaft 12, which may vary, can be seen. For example, the elongate shaft 12 may be formed from a plurality of layers such as an inner layer or liner 26, a reinforcing member or layer 28, and an outer layer 30. The inner layer 26 may include a lubricious material such as polytetrafluoroethylene. Other materials including those disclosed herein may be utilized. The reinforcing layer 28 may include a braid, a coil, both a braid and a coil, and/or the like. In some instances, the reinforcing layer 28 may include a slotted tube (e.g., a slotted hypotube, a slotted nickel-titanium alloy tube, etc.). The outer layer 30 may include a polyether block amide. Other materials including those disclosed herein may be utilized. In at least some instances, the outer layer 30 may include a plurality of different sections where more distal sections have an increased flexibility and/or lower durometer relative to more proximal sections. For example, a distal section of the outer layer 30 adjacent to the distal end region 14 of the elongate shaft 12 may include a relatively flexible material and a proximal region of the outer layer 30 (e.g., which may be along the proximal end region 16 of the elongate shaft 12) may include a less flexible material.

A low friction tube 22 may be disposed within the elongate shaft 12. The low friction tube 22 may comprise a tube formed from a lubricious material. In some instances, the low friction tube 22 may include an inner (e.g., lubricious) layer, a reinforcing layer, and another layer. Other forms are contemplated. A lumen 32 may be disposed centrally within the elongate shaft 12 for performing primary (e.g., left atrial appendage occlusion) or secondary intervention (e.g., PVI and/or a PVI+ maneuver) or both. The low friction tube 22 may resemble a small/miniature catheter shaft and may be disposed along the inner wall of the elongate shaft 12. Alternatively, the low friction tube 22 may be integrated between the inner layer 26 and the reinforcing layer 28 of the elongate shaft 12. In some instances, the low friction tube 22 may be formed from a single layer of lubricious material or a number of layers. For example, the low friction tube 22 may include an inner lubricious layer (e.g., formed from polytetrafluoroethylene or the like), a reinforcing layer, and an outer layer formed from polyether block amide. Other materials including those disclosed herein may be utilized. As indicated above, the steering wire 24 may be disposed within the low friction tube 22.

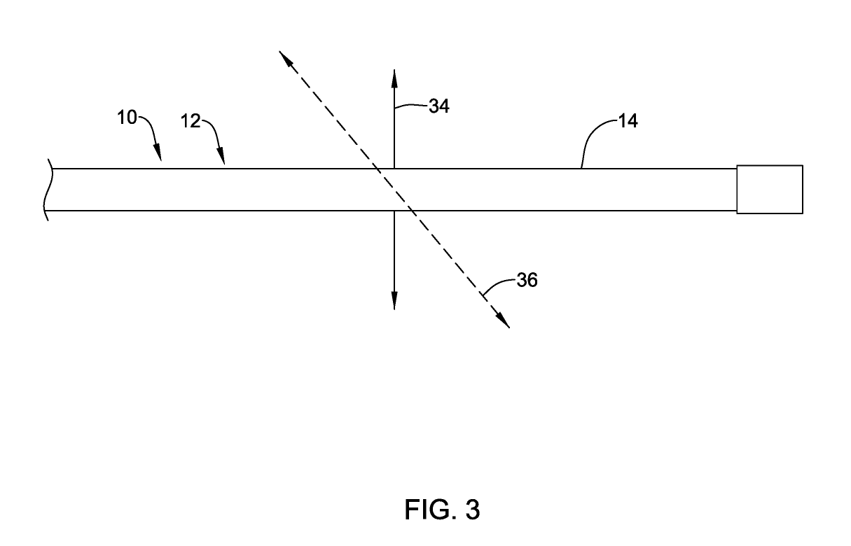

As indicated herein, some interventions may benefit from the ability of a medical device to bend in a tighter bend radius, for example to allow for reaching pulmonary veins and/or the like. Additionally, some interventions may benefit from the ability of a device to have a higher sheath force transmission and/or reach for enhancing positioning of the device in or adjacent to a left atrial appendage. The deflectable medical device 10 may have the ability for both bending in a tighter bend radius as well as higher sheath force transmission and/or reach. To help appreciate the properties of the deflectable medical device 10, FIG. 3 illustrates the deflectable medical device 10 and schematically represents that the elongate shaft 12 may be configured to be bent/steered/deflected in a number of different directions. In this example, the distal end region 14 of the elongate shaft 12 may be steerable in a first plane 34 and/or a second plane 36. The planes 34, 36 may be opposite one another, arranged orthogonally relative to one another, or otherwise be offset/intersecting (e.g., non-parallel at an angle, for example, between 0 and 90 degrees) relative to one another.

In at least some instances, the elongate shaft 12 may have differing bending characteristics when bending/steering the elongate shaft 12 in different directions. This may include different bending characteristics when bending/steering the elongate shaft 12 in different directions within the same plane (e.g., the first plane 34 or the second plane 36), different bending characteristics when bending/steering the elongate shaft 12 in different directions within the different planes (e.g., the first plane 34 or the second plane 36), or both. For example, the elongate shaft 12 may be configured to bend with a tighter bending radius when bending the elongate shaft 12 in a first direction within the first plane 34. When bending the elongate shaft 12 in a second or different direction (e.g., an opposite direction) in the first plane 34, the elongate shaft 12 may be configured to have higher sheath force transmission and/or reach. In other instances, the elongate shaft 12 may be configured to bend with a tighter bending radius when bending the elongate shaft 12 in a first direction and/or a second direction within the first plane 34. When bending the elongate shaft 12 in a third and/or fourth direction in the second plane 36, the elongate shaft 12 may be configured to have higher sheath force transmission and/or reach. Other arrangements and/or configurations are contemplated.

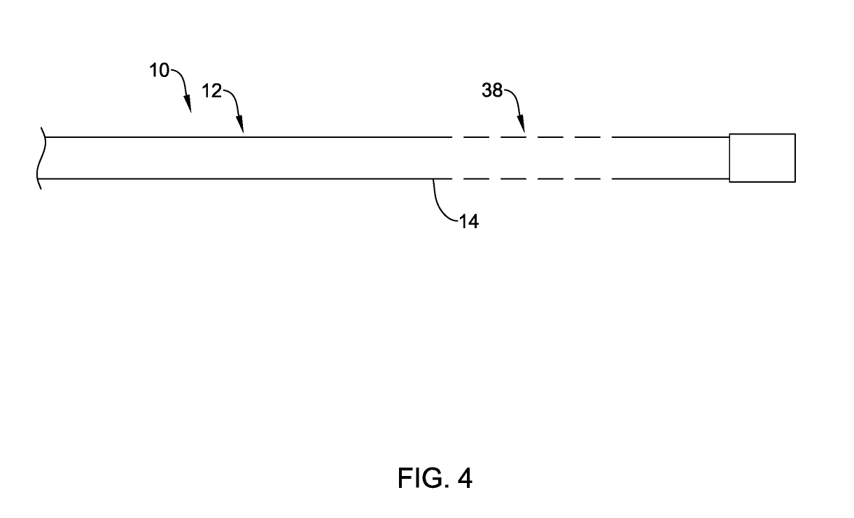

In order to provide the elongate shaft 12 with the desirable bending characteristics, the elongate shaft 12 may have material changes along one or more discrete regions of the shaft. For example, FIG. 4 schematically illustrates a discrete region 38 of the elongate shaft 12. In this example, the discrete region 38 is disposed along the distal end region 14 of the elongate shaft 12. However, this is not intended to be limiting as the discrete region 38 may disposed along essentially any suitable portion of the elongate shaft 12.

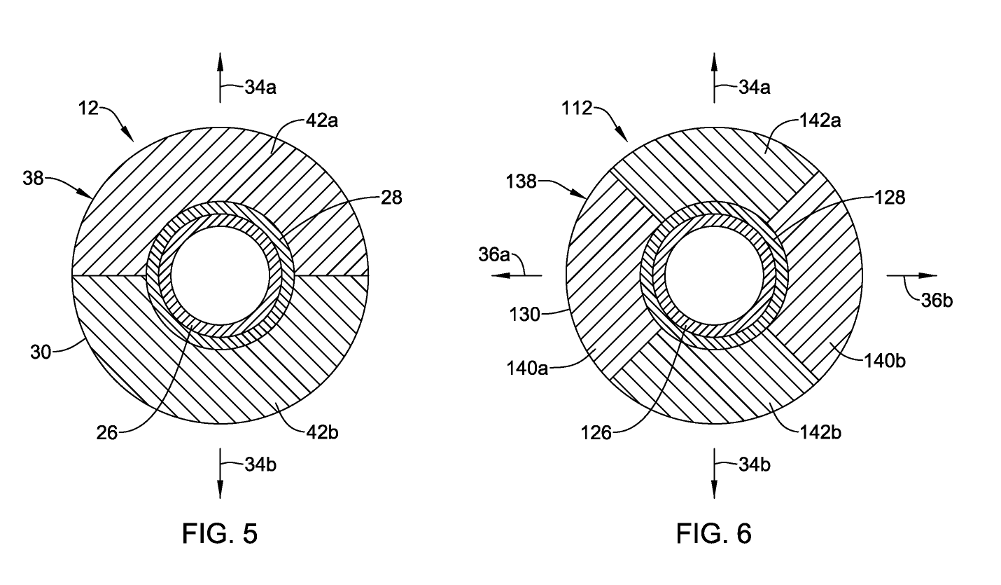

Along the discrete region 38, material of the elongate shaft 12, for example the outer layer 30, may be replaced by a polymer material (e.g., a different polymer material) with the desired properties so as to provide the elongate shaft 12 with the desired bending characteristics. When doing so, the “replacement” polymer material may define one or more polymer regions along the elongate shaft. For example, FIG. 5 is a cross-sectional view of the elongate shaft 12 along the discrete region 38. Here it can be seen that the outer layer 30 may have a first polymer region 42a and a second polymer region 42b. In some instances, material of the outer layer 30 may be removed and then replaced by a polymer material (e.g., a first polymer material) to define the first polymer region 42a. The second polymer region 42b may be made of the same, original material of the outer layer 30 or the material of the outer layer 30 may be removed and then replaced by a polymer material (e.g., a second polymer material) to define the second polymer region 42b.

In some instances, the elongate shaft 12 may have different bending characteristic when bending in a first direction (e.g., represented in FIG. 5 by an upward arrow and reference number 34a) and a second direction (e.g., represented in FIG. 5 by a downward arrow and reference number 34b). For example, the material of the first polymer region 42a may be configured to provide the elongate shaft 12 with a first stiffness when bending the elongate shaft 12 in the first direction 34a. The material of the second polymer region 42b may be configured to provide the elongate shaft 12 with a second stiffness, different from the first stiffness, when bending the elongate shaft 12 in the second direction 34b. In some instances, the material of the first polymer region 42a may have a lower stiffness (e.g., relative to the material of the second polymer region 42b and/or other portions of the outer layer 30) such that the elongate shaft 12 is configured to bend with a tighter bend radius when bending/steering the elongate shaft 12 in the first direction 34a. This may also be understood as the first polymer region 42a having a first radius of curvature when bending/steering the elongate shaft 12 in the first direction 34a and a second radius of curvature different from (e.g., larger than) the first radius of curvature bending/steering the elongate shaft 12 in the second direction 34b.

The material of the second polymer region 42b may have a higher stiffness (e.g., relative to the material of the first polymer region 42a and/or other portions of the outer layer 30) such that the elongate shaft 12 is configured to have higher sheath force transmission and/or reach when bending/steering the elongate shaft 12 in the second direction 34b. The materials for the first polymer region 42a and the second polymer region 42b may vary and may include suitable materials such as those disclosed herein. In some instances, more than one material and/or polymer blends may be utilized. Some examples may include the first polymer region 42a including a 35D polyether block amide (e.g., 35D PEBAX) and the second polymer region 42b including a 63D polyether block amide (e.g., 63D PEBAX). In another example, the first polymer region 42a may include nylon 12 and the second polymer region 42b may include a 35D polyether block amide (e.g., 35D PEBAX). In some instances, the material of the first polymer region 42a may be disposed along the elongate shaft 12 by removing/ablating (e.g., laser ablating) a portion of the outer layer 30 along the discrete region 38 and then adding replacement polymer material to the elongate shaft 12 (e.g., which may include a thermal process such reflowing). Adding material may include adding material after forming (e.g., via reflow) the outer layer 30, adding material prior to forming (e.g., via reflow) the outer layer 30, and/or the like. The added material may be added as singular layer of a singular material. Alternatively, adding materials may include adding a plurality of layers of material and/or a plurality of materials (e.g., via a dual or co-extrusion process, and/or the like). It should be noted that in order to take advantage of the desirable bending characteristics of the elongate shaft 12, the steering wires 24 may be arranged so that the steering wires 24 can be used to steer/bend the elongate shaft in first direction and the second direction and/or otherwise arranged so as to provide capability to bend in directions in order to take advantage of the desired bending characteristics.

In some of these and in other instances, a portion of outer layer 30 may be removed along the discrete region 38. If the reinforcing layer 28 includes a braid, at least some of the braid wires may be welded, soldered, and/or otherwise secured to one another. This may enhance the stiffness. The portions of the reinforcing layer 28 may be arranged so as to be disposed opposite the portions of the elongate shaft 12 that are configured to bend with a tighter bend radius. For example, in instances where the first polymer region 42a is configured to bend with a tighter bend radius, portion of the reinforcing layer 28 opposite the first polymer region 42a (e.g., portions adjacent to the second polymer region 42b) may include braid wires that are welded, soldered, and/or otherwise secured to one another. In some instances, this may allow for the outer layer 30 along only one side of the elongate shaft 12 to be replaced by a stiffer material. Alternatively, a slightly less stiff material may be utilized along the second polymer region 42b in order to achieve the same desired higher sheath force transmission and/or reach.

While bending characteristics may be altered in one plane, arrangements are contemplated where bending characteristics are altered for multiple planes and/or when bending in multiple/different directions. For example, FIG. 6 illustrates another example elongate shaft 112 that may be similar in form and function to other elongate shafts disclosed herein. The elongate shaft 112 may include an inner layer 126, a reinforcing layer 128, and an outer layer 130. Along a discrete region 138, a portion of the material of the outer layer 130 may be replaced by a first polymer material to define a first polymer region 142a, a portion of the material of the outer layer 130 may be replaced by a second polymer material to define a second polymer region 142b, a portion of the material of the outer layer 130 may be replaced by a third polymer material to define a third polymer region 140a, and a portion of the material of the outer layer 130 may be replaced by a fourth polymer material to define a fourth polymer region 140b.

The first polymer region 142a and the second polymer region 142b may be disposed along opposite sides (e.g., within the same plane) of the elongate shaft 112 and may provide the elongate shaft 112 with different bending characteristics. For example, the material of the first polymer region 142a may have a lower stiffness (e.g., relative to the material of the second polymer region 142b and/or other portions of the outer layer 130) such that the elongate shaft 112 is configured to bend with a tighter bend radius when bending/steering the elongate shaft 112 in the first direction 34a. The material of the second polymer region 142b may have a higher stiffness (e.g., relative to the material of the first polymer region 142a and/or other portions of the outer layer 130) such that the elongate shaft 112 is configured to have higher sheath force transmission and/or reach when bending/steering the elongate shaft 112 in the second direction 34b.

The third polymer region 140a and the fourth polymer region 140b may be disposed along opposite sides (e.g., within the same plane that is substantially orthogonal to the plane that the first polymer region 142a and the second polymer region 142b) of the elongate shaft 112 and may provide the elongate shaft 112 with different bending characteristics. For example, the material of the third polymer region 140a may have a lower stiffness (e.g., relative to the material of the fourth polymer region 140b and/or other portions of the outer layer 130) such that the elongate shaft 112 is configured to bend with a tighter bend radius when bending/steering the elongate shaft 112 in the first direction 36a. The material of the fourth polymer region 140b may have a higher stiffness (e.g., relative to the material of the third polymer region 140a and/or other portions of the outer layer 130) such that the elongate shaft 112 is configured to have higher sheath force transmission and/or reach when bending/steering the elongate shaft 112 in the second direction 36b.

In at least some instances, the first polymer region 142a and the second polymer region 142b may be formed from materials that are less stiff than those materials used for the third polymer region 140a and the fourth polymer region 140b. This may allow the elongate shaft 112 to have a tighter bend radius and/or require less force to deflect when deflected/steered in directions 34a, 34b relative to bending in directions 36a, 36b. The elongate shaft 112 may be configured to have higher sheath force transmission and/or reach when bending/steering the elongate shaft 112 in directions 36a, 36b relative to bending in directions 34a, 34b.

Some example materials may include the first polymer region 142a (and/or the third polymer region 140a) including a 35D polyether block amide (e.g., 35D PEBAX) and the second polymer region 142b (and/or the fourth polymer region 140b) including a 63D polyether block amide (e.g., 63D PEBAX). In another example, the first polymer region 142a (and/or the third polymer region 140a) may include nylon 12 and the second polymer region 142b (and/or the fourth polymer region 140b) may include a 35D polyether block amide (e.g., 35D PEBAX). In another example, the first polymer region 142a and the second polymer region 142b are formed from a 35D polyether block amide (e.g., 35D PEBAX) or nylon 12, and the third polymer region 140a and the fourth polymer region 140b are formed from a 63D polyether block amide (e.g., 63D PEBAX) or a 35D polyether block amide (e.g., 35D PEBAX). In some instances, the first polymer region 142a and the second polymer region 142b are formed from the same materials, and the third polymer region 140a and the fourth polymer region 140b are formed from the same materials (e.g., which may be different from the materials used for the first polymer region 142a and the second polymer region 142b). Of course, other arrangements are contemplated where the materials differ.

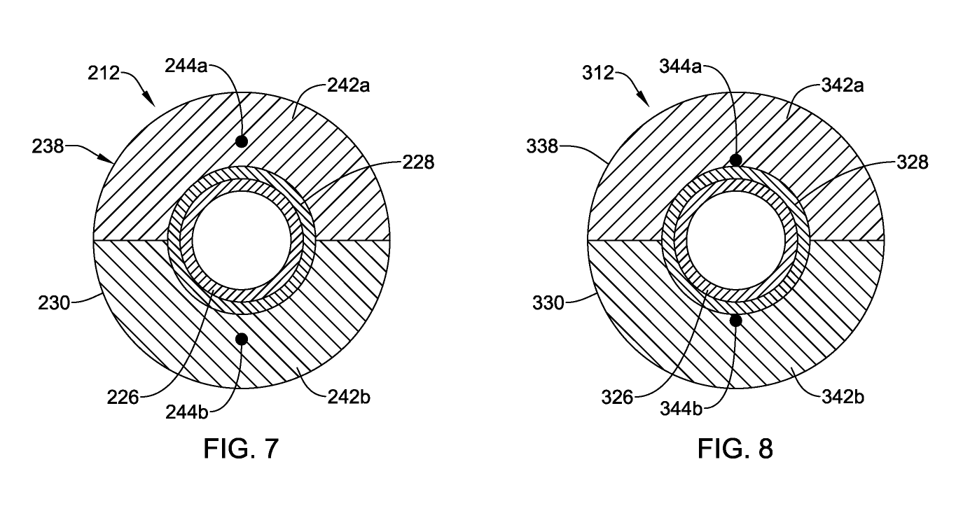

In some instances, a stiffening member may be added to enhance the stiffness of the elongate shaft. For example, FIG. 7 illustrates another example elongate shaft 212 that may be similar in form and function to other elongate shafts disclosed herein. The elongate shaft 212 may include an inner layer 226, a reinforcing layer 228, and an outer layer 230. Along a discrete region 238, a portion of the material of the outer layer 230 may be replaced by a first polymer material to define a first polymer region 242a and a portion of the material of the outer layer 230 may be replaced by a second polymer material to define a second polymer region 242b. One or more stiffening members, such as stiffening members 244a, 244b, may be disposed along the first polymer region 242a and/or the second polymer region 242b. While both the first polymer region 242a and the second polymer region 242b are shown having a stiffening member 244a, 244b, embodiments are contemplated where only one of the polymer regions (e.g., the second polymer region 242b) includes the stiffening member (e.g., the stiffening member 244b) and the other polymer region (e.g., the first polymer region 242a) is free of a stiffening member (e.g., the stiffening member 244a). As the name suggests, the stiffening member(s) 244a, 244b may have an enhanced stiffness so as to provide the first polymer region 242a, the second polymer region 242b, or both with increased stiffness. For example, the stiffening member(s) 244a, 244b may be formed from a stainless-steel wire or the like. The stiffening member(s) 244a, 244b may extend the length of the elongate shaft 212 or may extend only the length of the discrete region 238. Although FIG. 7 depicts one stiffening member(s) 244a, 244b in each of the first polymer region 242a and the second polymer region 242b, multiple stiffening members may be present in each portion to impact the overall stiffness characteristics. Other materials are contemplated including those materials disclosed herein.

The first polymer region 242a and the second polymer region 242b may be disposed along opposite sides (e.g., within the same plane) of the elongate shaft 212 and may provide the elongate shaft 212 with different bending characteristics, for example bending characteristics similar to those described herein. For example, the material of the first polymer region 242a may have a lower stiffness (e.g., relative to the material of the second polymer region 242b and/or other portions of the outer layer 230) such that the elongate shaft 212 is configured to bend with a tighter bend radius when bending/steering the elongate shaft 212 in a first direction (e.g., similar to the first direction 34a). The material of the second polymer region 242b may have a higher stiffness (e.g., relative to the material of the first polymer region 242a and/or other portions of the outer layer 230) such that the elongate shaft 212 is configured to have higher sheath force transmission and/or reach when bending/steering the elongate shaft 212 in a second direction (e.g., similar to the second direction 34b). The stiffening member(s) 244a, 244b may be used to further enhance the stiffness. For example, the stiffening member 244b may be disposed along the second polymer region 242b (and, in some instances, the first polymer region 242a may be free of a stiffening member). The stiffening members 244a, 244b may extend along the distal region of the elongate shaft 212 or along the entire length of the elongate shaft 212.

FIG. 8 illustrates another example elongate shaft 312 that may be similar in form and function to other elongate shafts disclosed herein. The elongate shaft 312 may include an inner layer 326, a reinforcing layer 328, and an outer layer 330. Along a discrete region 338, a portion of the material of the outer layer 330 may be replaced by a first polymer material to define a first polymer region 342a and a portion of the material of the outer layer 330 may be replaced by a second polymer material to define a second polymer region 342b. One or more stiffening members, such as stiffening members 344a, 344b, may be disposed along the first polymer region 342a and/or the second polymer region 342b. While both the first polymer region 342a and the second polymer region 342b are shown having a stiffening member 344a, 344b, embodiments are contemplated where only one of the polymer regions (e.g., the second polymer region 342b) includes the stiffening member (e.g., the stiffening member 344b) and the other polymer region (e.g., the first polymer region 342a) is free of a stiffening member (e.g., the stiffening member 344a).

The first polymer region 342a and the second polymer region 342b may be disposed along opposite sides (e.g., within the same plane) of the elongate shaft 312 and may provide the elongate shaft 312 with different bending characteristics, for example bending characteristics similar to those described herein. For example, the material of the first polymer region 342a may have a lower stiffness (e.g., relative to the material of the second polymer region 342b and/or other portions of the outer layer 330) such that the elongate shaft 312 is configured to bend with a tighter bend radius when bending/steering the elongate shaft 312 in a first direction (e.g., similar to the first direction 34a). The material of the second polymer region 342b may have a higher stiffness (e.g., relative to the material of the first polymer region 342a and/or other portions of the outer layer 330) such that the elongate shaft 312 is configured to have higher sheath force transmission and/or reach when bending/steering the elongate shaft 312 in a second direction (e.g., similar to the second direction 34b). The stiffening member(s) 344a, 344b may be used to further enhance the stiffness. For example, the stiffening member 344b may be disposed along the second polymer region 342b (and, in some instances, the first polymer region 342a may be free of a stiffening member).

In the example shown in FIG. 8, the stiffening member(s) 344a, 344b are shown disposed adjacent to the reinforcing layer 328. This may include bonding the stiffening member(s) 344a, 344b to the reinforcing layer 328, weaving the stiffening member(s) 344a, 344b with the reinforcing layer 328, and/or the like. The stiffening members 344a, 344b may extend along the distal region of the elongate shaft 312 or along the entire length of the elongate shaft 312.

The materials that can be used for the various components of the deflectable medical device 10 may include those commonly associated with medical devices. For simplicity purposes, the following discussion makes reference to the elongate shaft 12. However, this is not intended to limit the devices and methods described herein, as the discussion may be applied to other similar tubular members and/or components of tubular members or devices disclosed herein.

The elongate shaft 12 and/or other components of the deflectable medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as VESTAMID®, GRILAMID® available from EMS American Grilon, and/or the like), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the deflectable medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the deflectable medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the deflectable medical device 10 to achieve the same result.

In some embodiments, a degree of Magnetic Resonance Imaging (MRI) compatibility is imparted into the deflectable medical device 10. For example, the deflectable medical device 10, or portions thereof, may be made of a material that does not substantially distort the image and create substantial artifacts (e.g., gaps in the image). Certain ferromagnetic materials, for example, may not be suitable because they may create artifacts in an MRI image. The deflectable medical device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A deflectable medical device, comprising:
an elongate shaft having a distal end region;
wherein the distal end region has a first polymer region defined along a first side, the first polymer region including a first polymer;
wherein the distal end region has a second polymer region defined along a second side, the second polymer region including a second polymer;
wherein the first polymer has a different stiffness than the second polymer; and
wherein the distal end region is configured to have a first radius of curvature along the first polymer region when bending in a first direction and a second radius of curvature along the second polymer region when bending in a second direction.

2. The deflectable medical device of claim 1, wherein the first direction and the second direction lie within a first plane.

3. The deflectable medical device of claim 1, wherein the first direction lies within a first plane and the second direction lies within a second plane different from the first plane.

4. The deflectable medical device of claim 1, wherein the elongate shaft includes an inner layer, a reinforcing layer, and an outer layer.

5. The deflectable medical device of claim 4, wherein the first polymer region is defined by replacing a first discrete section of the outer layer with the first polymer.

6. The deflectable medical device of claim 5, wherein the second polymer region is defined by replacing a second discrete section of the outer layer with the second polymer.

7. The deflectable medical device of claim 4, wherein further comprising a stiffening member disposed along the first polymer region.

8. The deflectable medical device of claim 7, wherein the stiffening member engages the reinforcing layer.

9. The deflectable medical device of claim 1, wherein the first polymer region includes a plurality of polymers.

10. The deflectable medical device of claim 1, wherein the first polymer region is formed by laser ablating an ablated region of the elongate shaft and disposing the first polymer along the ablated region.

11. A deflectable medical device, comprising:
an elongate shaft having a distal end region;
wherein the elongate shaft includes an inner layer, a reinforcing layer, and an outer layer;
wherein a first discrete section of the outer layer along the distal end region is replaced by a first polymer to define a first polymer region;
wherein a second discrete section of the outer layer along the distal end region is replaced by a second polymer to define a second polymer region;
wherein the first polymer has a different stiffness than the second polymer; and
wherein the distal end region is configured to have a first radius of curvature along the first polymer region when bending in a first direction and a second radius of curvature along the second polymer region when bending in a second direction.

12. The deflectable medical device of claim 11, wherein the first direction and the second direction lie within a first plane.

13. The deflectable medical device of claim 11, wherein the first direction lies within a first plane and the second direction lies within a second plane different from the first plane.

14. The deflectable medical device of claim 11, wherein further comprising a stiffening member disposed along the first polymer region.

15. The deflectable medical device of claim 14, wherein the stiffening member is disposed along the first polymer region.

16. The deflectable medical device of claim 14, wherein the stiffening member engages the reinforcing layer.

17. The deflectable medical device of claim 11, wherein the first polymer region includes a plurality of polymers.

18. The deflectable medical device of claim 11, wherein the first discrete section of the outer layer is laser ablated from the outer layer.

19. A method for forming a deflectable medical device, the method comprising:
replacing a first discrete section of an outer layer of an elongate shaft with a first polymer to define a first polymer region;
wherein the elongate shaft includes a distal end region, an inner layer, a reinforcing layer, and the outer layer;
wherein the first discrete section of the outer layer is disposed along the distal end region of the elongate shaft;
replacing a second discrete section of the outer layer along the distal end region with a second polymer to define a second polymer region;
wherein the first polymer has a different stiffness than the second polymer; and
wherein the distal end region is configured to have a first radius of curvature along the first polymer region when bending in a first direction and a second radius of curvature along the second polymer region when bending in a second direction.

20. The method of claim 19, further comprising disposing a stiffening member adjacent to the first polymer region.
